(19) United States
(12) Patent Application Publication
(10) Pub. No.: US 2019/0275003 A1
(43) Pub. Date: Sep. 12, 2019

(54) ENCAPSULATION OF PHOSPHODIESTERASE INHIBITORS TO TREAT ALCOHOLIC LIVER DISEASE

(71) Applicant: University of Louisville Research Foundation, Inc., Louisville, KY (US)

(72) Inventors: Claudio Maldonado, Louisville, KY (US); Craig McClain, Louisville, KY (US); Shirish S. Barve, Louisville, KY (US); Leila Gobejishvili, Louisville, KY (US); Philip Bauer, Louisville, KY (US)

(21) Appl. No.: 16/347,908

(22) PCT Filed: Nov. 8, 2017
(86) PCT No.: PCT/US2017/060642

§ 371 Date: May 7, 2019

Related U.S. Application Data
(60) Provisional application No. 62/419,114, filed on Nov. 8, 2016.

(51) Int. Cl. A61K 31/4015; A61K 9/127; A61K 47/26; A61K 47/24; A61P 1/16

(52) U.S. Cl. CPC A61K 31/4015; A61K 9/127; A61P 1/16; A61K 47/24; A61K 47/26

(57) ABSTRACT

Provided herein are compositions for treating liver inflammation. The compositions include a biologically active phosphodiesterase 4 (PDE4) inhibitor and a liposome, the liposome encapsulating the PDE4 inhibitor. The liposome may be anionic and include both neutrally charged and negatively charged phospholipids. Encapsulation of the PDE4 inhibitor in the liposome facilitates targeted delivery to the liver while limiting access to the central nervous system.

### RELATED APPLICATIONS

This application claims the benefit of U.S. Provisional Application Ser. No. 62/419,114, filed Nov. 8, 2016, the entire disclosure of which is incorporated herein by this reference.

### GOVERNMENT INTEREST

This invention was made with government support under grant numbers R43AA021331 and R44AA021331 awarded by the National Institutes of Health (NIH). The government has certain rights in the invention.

### TECHNICAL FIELD

The presently-disclosed subject matter generally relates to methods and products for encapsulation of, delivery of, and/or treatment with rolipram. In particular, certain embodiments of the presently-disclosed subject matter relate to fusogenic lipid vesicles (FLVs), encapsulation of rolipram therein, and treatment methods using the encapsulated rolipram.

### BACKGROUND

Despite extensive research, alcohol abuse remains one of the most common causes of acute and chronic liver disease in the United States and worldwide. In Western countries, up to 50% of end-stage liver disease cases have alcohol as a major etiologic factor, with excessive alcohol consumption being the third leading preventable cause of death in the United States. Alcohol-related deaths, excluding accidents/homicides, accounted for 22,073 deaths in the United States in 2006. In particular, alcoholic liver disease (ALD), which accounted for 13,000 of those 22,073 alcohol related deaths, remains a major cause of liver related mortality in the U.S. The mortality of this liver disease is more than that of many major forms of cancer (e.g., breast, colon, and prostate). For example, survival rates of 5 and 10 years for alcoholic cirrhosis have been reported by some groups at 23% and 7%, respectively. Projections indicate that the annual costs of this problem exceed 2.5 billion dollars in the U.S. However, as no drug therapy has yet been approved by the Food and Drug Administration (FDA) for any stage of ALD, the only currently accepted approach to ALD by most physicians is abstinence and optimal nutrition.

One major feature of ALD includes abnormal cytokine metabolism. For example, dysregulated tumor necrosis factor-α (TNF) metabolism has been identified in severe alcoholic hepatitis (AH). It was observed that peripheral monocytes (which produce the overwhelming majority of systemic circulating TNF and are a surrogate marker for Kupffer cells) from AH patients spontaneously produced significantly more TNF in response to an endotoxin (LPS) stimulus. Additionally, increased serum TNF concentrations in ALD were reported and values correlated with disease severity and mortality. More specifically, elevated serum concentrations of TNF and TNF-inducible proinflammatory cytokines/chemokines, such as interleukin (IL)-8 and IL-18, have been reported in patients with alcoholic hepatitis and/or cirrhosis. These elevated levels correlate with markers of the acute phase response, reduced liver function, and poor clinical outcome.

While several pharmacological therapies have been tested in patients with alcoholic liver disease, to date, none has shown consistent improvement in the course of alcoholic hepatitis with and without superimposed cirrhosis. For example, although anti-TNF antibodies and soluble receptors are FDA approved and used clinically with excellent results for chronic inflammatory diseases such as Crohn's disease, rheumatoid arthritis, and others, in the context of liver disease, a complete inhibition of TNF could potentially impair liver regeneration with detrimental consequences. Initial small trials of anti-TNF antibody in AH seemed positive, however, a large French multicenter trial using anti-TNF antibody plus prednisone, was stopped due to increased mortality. Also, a U.S. multicenter trial with a TNF-soluble receptor in AH showed no benefit. Furthermore, potential side effects and expense are associated with anti-TNF therapy, and these effects may limit their use in ALD.

As an alternative to antibodies and soluble receptors, it has been shown that LPS-inducible TNF expression by monocytes/macrophages is critically regulated by the intracellular cyclic adenosine monophosphate (cAMP) levels. The instant inventors recently demonstrated that chronic ethanol exposure of monocytes/macrophages (including Kupffer cells) decreases both basal and LPS-stimulated cyclic adenosine monophosphate (cAMP) levels by up-regulating phosphodiesterase (PDE) 4 expression, which leads to the enhancement of LPS-inducible TNF production. Of the PDE4 A, B and D isoforms predominantly expressed in monocytes/macrophages, it has been established that PDE4B is involved in LPS-induced signaling mediated by TLR4 and is essential for LPS-induced TNF expression.

Phosphodiesterase (PDE) inhibitors, which increase cAMP levels, have been extensively studied and have been demonstrated to effectively inhibit TNF production in vivo and in vitro. Importantly, PDE4 inhibitors also have also been demonstrated to up-regulate cytokine IL-10, which has anti-inflammatory and anti-fibrotic properties. Accordingly, PDE inhibitors may represent possible therapies for chronic inflammatory processes. For example, in human clinical studies, Pentoxifylline (PTX), a non-specific PDE inhibitor, decreased mortality in patients with AH. PTX has also been shown to attenuate liver injury and fibrosis in several animal models. In addition, the instant inventors recently showed the pathogenic role of PDE4 enzymes in the development of cholestatic liver injury and fibrosis, along with significant protection by PDE4 specific inhibitor rolipram. Further, the instant inventors have reported that rolipram-mediated inhibition of PDE4 significantly down-regulates LPS-inducible TNF. However, the therapeutic use of PDE4 inhibitors (including rolipram) to treat alcohol-induced hepatic inflammation and liver disease is precluded by severe dose-associated side effects including severe nausea and emesis caused by the inhibition of PDE4 in the central nervous system (CNS) and/or the increased cAMP levels in the CNS. The systemic therapeutic doses of rolipram or other PDE4 inhibitors required to reduce TNF expression in ALD will consistently produce side effects.

Accordingly, there exists a need for products and methods that effectively treat ALD without the prohibitive side effects of current treatments.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a composition for treating liver inflammation, the composition comprising a biologically active phosphodiesterase 4 (PDE4) inhibitor. In some embodiments, the composition further comprises a liposome encapsulating the inhibitor rolipram. In one embodiment, the liposome is an anionic liposome. In another embodiment, the liposome comprises negatively charged phospholipids and neutrally charged phospholipids. In a further embodiment, a molar ratio of the neutrally charged phospholipids to the negatively charged phospholipids is between 5:1 and 1:1. In some embodiments, the negatively charged phospholipids are phosphatidic acid (PA). In some embodiments, the neutrally charged phospholipids are phosphatidylcholine (PC). In certain embodiments, the liposome comprises 1,2-Dioleoyl-sn-glycerol-3-phosphocholine (DOPC) and 1-palmitoyl-2-oleol-sn-glycerol-3-phosphate (POPA). Additionally or alternatively, in some embodiments, the composition further comprises an excipient. In one embodiment, the excipient is sucrose octaacetate.

Also provided herein, in some embodiments, is a composition for treating liver inflammation, the composition comprising a phosphodiesterase 4 (PDE4) inhibitor and a liposome, the liposome encapsulating the PDE4 inhibitor. In one embodiment, the liposome comprises negatively charged phospholipids and neutrally charged phospholipids. In another embodiment, the liposome is an anionic liposome. In a further embodiment, a molar ratio of the neutrally charged phospholipids to the negatively charged phospholipids is between 5:1 and 1:1. In some embodiments, the negatively charged phospholipids are phosphatidic acid (PA). In some embodiments, the neutrally charged phospholipids are phosphatidylcholine (PC). In certain embodiments, the liposome comprises 1,2-Dioleoyl-sn-glycerol-3-phosphocholine (DOPC) and 1-palmitoyl-2-oleol-sn-glycerol-3-phosphate (POPA).

In some embodiments, the PDE4 inhibitor is a biologically active analogue of rolipram. In some embodiments, the PDE4 inhibitor is selected from the group consisting of rolipram, apremilast, crisaborole, roflumilast, cilomilast, piclamilast, ibudilast, and lirimilast. In some embodiments, the composition further comprises an excipient. In one embodiment, the excipient is sucrose octaacetate.

### DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-B show graphs showing that chronic alcohol exposure significantly attenuates intracellular cAMP levels in macrophages and Kupffer cells. (A) After 6 week-alcohol exposure (25 mM), macrophages were stimulated with LPS (100 ng/ml) and collected after 3 hours for cAMP measurements. *P<0.01 compared with un-stimulated control and **P<0.01 compared with LPS-stimulated control. (B) cAMP levels were measured in Kupffer cells from control (n=4) and alcohol fed (n=4) rats. *P<0.01 compared with control. Data are presented as mean±SD. LPS=endotoxin.

FIG. 2 shows graphs illustrating that chronic alcohol exposure of RAW cells markedly up-regulates LPS inducible PDE4B and TNF mRNA expression. Cells were stimulated with LPS (100 ng/mL) and PDE4B and TNF mRNA was quantified using real time PCR. *P<0.01 compared with LPS-stimulated control. TNF=tumor necrosis factor-alpha, LPS=endotoxin, PDE4B=phosphodiesterase 4B.

FIG. 3 shows a graph illustrating TNF expression under various conditions. PBMCs isolated from a healthy individual were pre-treated with rolipram (1, 5, and 10 μM) for 30 minutes and further stimulated with LPS (1 μg/ml) for 6 hours. Cell-free supernatants were analyzed for TNF expression by ELISA. Rol=rolipram. TNF=tumor necrosis factor-alpha, FLVs=fusogenic lipid vesicles.

FIG. 4 shows a graph illustrating the encapsulation efficiency percent (EE %) of rolipram (0.75 mg/mL) in FLVs is dependent on the lipid concentration in buffer. As the lipid concentration of DOPC/POPA formulated FLVs increased from 5 to 15 mg/mL the EE % also increased, but not proportionally. FLVs=fusogenic vesicles.

FIGS. 5A-C show the encapsulation efficiency percent of rolipram when 10 (A), 12.5 (B) and 15 mg/mL of FLV lipid (C) was hydrated with buffer containing 0.75 mg/mL of rolipram and diluted in water five or 10 times. The percentage of free rolipram in buffer was calculated by measuring the amount of free drug in buffer after FLVs in solution were filtered out. More than 50% of drug remained encapsulated with either of the dilutions over time. The amount of rolipram associated with FLVs appeared to fluctuate over time but it stabilized at the end of 105 min. FLVs=fusogenic vesicles.

FIG. 6 shows a graph illustrating the encapsulation efficiency percent (EE %) of rolipram (0.75 mg/mL) in FLVs (10 mg/mL of lipid) when the excipients SOA and SAIB are used and diluted five or ten times. After dilution the rolipram EE % of FLVs was similar when either excipient or no excipient was utilized. FLVs=fusogenic lipid vesicles, SOA=Sucrose octaacetate, SAIB=acetate isobutyrate.

FIG. 7 shows a graph illustrating results from RAW cell assay experiments to determine the effects of the excipient SAIB. Cells were treated with SAIB (0.5 mg/mL), FLVs (10 mg/mL of lipid) with SAIB (FLV-SAIB) or FLVs-rolipram 0.75 mg/mL (FLV-Rol) without SAIB and stimulated with LPS (100 ng/mL) for 18 h. The effect of SAIB was evaluated by quantifying TNF production using an ELISA kit. SAIB alone appears to exert a beneficial effect, and FLVs with SAIB have a greater beneficial effect. FLVs=fusogenic lipid vesicles, LPS=endotoxin, SAIB=acetate isobutyrate.

FIGS. 8A-B show the fusion rate of FLVs-Rolipram to RAW cells and mouse aortic endothelial cells (MAECs) in the presence or absence of excipients in the vehicle. Using fluorescent-labeled FLVs and a Nanoparticle Tracking Analysis instrument to quantify FLV numbers, the fusion rate of vesicles to RAW cells was quantified over time. (A) Lipid vesicles formulated with DOPC/POPA (3:2) loaded with rolipram (DOPC:POPA-R) fused to RAW cells at a faster rate than DOPC vesicles loaded with rolipram (DOPC). (B) Lipid vesicles formulated with DOPC/POPA (3:2) loaded with rolipram and SOA (DOPC:POPA-R SOA) or SAIB (DOPC:POPA-R SAIB) fused at a faster rate to MAECs than DOPC-R. SAIB appeared to decrease the fusion rate of FLVs-rolipram. These studies demonstrated that DOPC:POPA-R FLVs fuse rapidly with different types of cells but at different rates. FLVs=fusogenic lipid vesicles, SOA=Sucrose octaacetate, SAIB=acetate isobutyrate.

FIG. 9 shows images illustrating distribution of DiR-labeled FLVs in mice before (baseline) i.v. infusion and after 10 min, 2 h and 72 h. Note: near-infrared dye is mostly localized to liver (based on x-ray image) after 10 min post infusion and from 2 h to 72 h DiR-labeled FLVs are mainly localized in liver. Negligible levels of signal were detected in brain or lungs. FLVs=fusogenic lipid vesicles.

FIG. 10 shows graphs illustrating serum cytokine levels for TNF, MCP-1, and hepatic TNF mRNA in rats. Wistar rats (mean weight of 250 g) received one of the following treatments: PBS (sham), empty FLVs, LPS only, and FLVs-rolipram at doses of 1, 2, and 3.3 mg/kg. FLVs-rolipram groups received an LPS injection 4 h after drug therapy. Serum cytokine levels for TNF, MCP-1, and hepatic TNF mRNA levels were measured 6 h after LPS injection. Data are presented as mean±SD. Rol=rolipram, FLV=fusogenic lipid vesicle, LPS=endotoxin, TNF=tumor necrosis factor-alpha, MCP-1=Monocyte chemoattractant protein-1, PBS=phosphate buffer saline. N=10 in each treatment group. **P<0.01, ***P<0.001.

FIG. 11 shows a graph illustrating serum ALT and AST after 10 h of FLV administration to evaluate FLV-lipid toxicity on the liver. No differences were found.

FIGS. 12A-B show graphs illustrating the effect of FLVs-rolipram on PDE4 activity. (A) PDE4 activity measured in liver tissue lysates. (B) PDE4 activity measured in brain tissue lysates. Note that hepatic PDE4 activity was decreased in liver up to 72 h but not in brain at any point. FLV=fusogenic lipid vesicle, Rol=rolipram, LPS=endotoxin, PDE4=phosphodiesterase 4. Data are presented as mean±SD. N=10 in each group. **P<0.01, ***P<0.001.

FIG. 13 shows graphs illustrating the effect of FLVs-rolipram on serum TNF (left) and MCP-1 (right) levels induced by LPS stimulus. Mice were pretreated with FLV-Rol 12 h before LPS administration and serum cytokines were measured 3 h post LPS. FLV-Rol pretreatment significantly attenuated LPS-inducible TNF and MCP-1 levels. Ctrl=control, Rol=rolipram, FLV=fusogenic lipid vesicle, LPS=endotoxin. Data are presented as mean±SD. N=10 in each group. *P<0.05, **P<0.01, ***P<0.001.

FIG. 14 shows a graph illustrating results from RAW cell assay experiments. Cells were treated with 0.73 mg/mL rolipram (Rol), FLVs only, or 0.73 mg/mL of FLVs-rolipram (FLVs-Rol) and stimulated with LPS (100 ng/mL) for 24 h. Control cells received treatment but no LPS. TNF in media was measured using an ELISA kit. UT=untreated cells, FLVs=fusogenic vesicles, LPS=endotoxin.

FIG. 15 shows a graph illustrating significant up regulation of hepatic PDE4 after ethanol feeding. PDE4B, C and D mRNA levels were measured in the livers of PF and AF mice. Data are presented as mean±SD, n=10, *P<0.05. PF=pair fed, AF=alcohol feeding, PDE4=Phosphodiesterase 4.

FIGS. 16A-B shows representative images of Oil Red O and CAE stained liver sections. (A) Oil Red O staining of liver sections to identify areas of steatosis. (B) CAE staining of liver sections to identify neutrophil infiltration (black arrows). Mice were fed Lieber-DeCarli alcohol or control liquid diet for 10 days and administered FLVs-rolipram (3.3 mg/kg bw). On day 11th mice were gavaged with 31.5% alcohol or maltose dextrin, and 6 h later livers were harvested. PF=pair fed, AF=alcohol feeding, FLV=fusogenic lipid vesicle, Rol=rolipram.

FIGS. 17A-B shows graphs illustrating hepatic caspase-3 activity and serum AST levels under various conditions. (A) Hepatic caspase 3 assay was performed to demonstrate the effect of FLVs-rolipram on alcohol induced hepatic apoptosis. (B) Serum AST levels demonstrating the effect of FLVs-rolipram on alcohol-induced liver injury. Mice were fed Lieber-DeCarli alcohol or control liquid diet for 10 days and administered FLVs-rolipram (3.3 mg/kg bw). On day 11th mice were gavaged with 31.5% alcohol or maltose dextrin and 6 h later livers were harvested. PF=pair-fed, AF=alcohol feeding, FLV=fusogenic lipid vesicle, Rol=Rolipram, AST=Aspartate transaminase. *P<0.05, **P<0.01.

FIG. 18 shows a graph illustrating serum endotoxin levels after alcohol-binge. Mice were fed Lieber-DeCarli alcohol or control liquid diet for 10 days and administered FLVs-rolipram (3.3 mg/kg bw). On day 11th mice were gavaged with 31.5% alcohol or maltose dextrin and 6 h later livers were harvested. PF=pair-fed, AF=alcohol feeding, FLV=fusogenic lipid vesicle, Rol=rolipram.

FIG. 19 shows a graph illustrating that FLVs-rolipram significantly affects LPS-inducible liver TNF and IL-10 mRNA levels in the liver. **P<0.01 compared to LPS alone. LPS=endotoxin, FLVs=fusogenic lipid vesicles.

FIGS. 20A-E shows graphs and images illustrating that FLVs-rolipram therapy markedly attenuated liver injury in a rat model of cholestatic liver injury or bile duct ligation (BDL) model. (A) H&E staining of sham. (B) H&E staining of BDL no treatment. (C) H&E staining of BDL+FLVs-rolipram therapy. (D) Graph showing AST levels. (E) Graph showing ALT Levels. FLVs=fusogenic lipid vesicles.

FIG. 21 shows a graph illustrating results from paired studies examining the effect of various doses of rolipram (1.6, 3.3 and 6.6 mg/kg bw) on anesthesia duration. *P<0.01 compared to its paired control time. Rol=rolipram.

FIG. 22 shows a graph illustrating results from Paired studies examining the effect of various doses of FLVs (55 and 110 mg/kg bw of lipid)-rolipram (1.6, 3.3 and 6.6 mg/kg bw) on anesthesia duration. FLVs-Rol=Fusogenic lipid vesicles-rolipram.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes products for encapsulation and/or delivery of one or more phosphodiesterase (PDE) inhibitors. In some embodiments, the products include lipid vesicles, such as fusogenic lipid vesicles (FLVs) and/or nanoliposomes (NLs), arranged and disposed for entrapment and/or encapsulation of one or more PDE inhibitors. In some embodiments, the lipid vesicles are arranged and disposed to target the PDE inhibitors to the liver and/or decrease or eliminate access of the PDE inhibitors to the central nervous system (CNS). For example, in one embodiment, the lipid vesicles target the PDE inhibitor(s) to the liver while limiting PDE access to the CNS. In another embodiment, the lipid vesicles facilitate and/or provide reduced PDE activity in the liver but not in the brain. In a further embodiment, the lipid vesicles decrease or eliminate CNS side effects of PDE inhibitors as compared PDE inhibitors alone.

In contrast to other drug-carrying liposomes that are designed and formulated to reduce uptake by the reticuloendothelial system (RES), which prolongs circulation time and the biological half-life of the drugs, the lipid vesicles described herein are arranged and disposed to fuse with cells and increase uptake by the RES (e.g., Kupffer cells), which shortens circulation time of the lipid vesicle-PDE inhibitor complex. Without wishing to be bound by theory, it is believed that the increased uptake of the lipid vesicle-PDE inhibitor complex by the RES decreases or eliminates free systemic circulation of the PDE inhibitor, which prevents or substantially prevents the PDE inhibitor from crossing the blood-brain barrier and reaching the brain. This decreases or eliminates the side effects induced by PDE inhibitors in the brain, such as severe nausea and emesis, thereby permitting and/or facilitating the use of PDE inhibitors in disease therapy.

The biodistribution of the lipid vesicles is determined, at least in part, by the lipid composition, charge, and/or vesicle size thereof. For example, in some embodiments, the lipid vesicles include a lipid composition configured to increase vesicle-to-cell fusion rates. In one embodiment, a charge of the phospholipid head group may be manipulated to create dissimilar regions in the lipid layer. In another embodiment, the lipid composition provides the lipid vesicle with an overall negative charge. In a further embodiment, the overall negative charge of the lipid vesicles facilitates and/or promotes vesicle-to-cell fusion. Additionally or alternatively, the overall negative charge of the lipid vesicle prevents or substantially prevents the vesicles from fusing with each other. These properties reduce or eliminate lipid vesicle fusion while transiting in circulating blood (i.e., unwanted lipid vesicle fusion), which reduces or eliminates systemic drug release from such unwanted fusion.

In certain embodiments, the lipid composition includes at least one neutrally charged phospholipid and at least one negatively charged phospholipid, at physiological pH. In one embodiment, the neutrally charged phospholipids include, but are not limited to, phosphocholines (PCs). In another embodiment, the negatively charged phospholipids include, but are not limited to, phosphatidic acids (PAs).

Suitable PCs include, but are not limited to, saturated PCs, such as 12:0 PC 1,2-dilauroyl-sn-glycero-3-phosphocholine (DLPC), 14:0 PC 1,2-dimyristoyl-sn-glycero-3-phosphocholine (DMPC), 16:0 PC 1,2-dipalmitoyl-sn-glycero-3-phosphocholine (DPPC), 18:1 (Δ9-Cis) PC 1,2-dioleoyl-sn-glycero-3-phosphocholine (DOPC), 20:1 (Δ11-Cis) PC 1,2-dieicosenoyl-sn-glycero-3-phosphocholine, or a combination thereof.

Suitable PAs include, but are not limited to, 16:0-18:1 PA 1-palmitoyl-2-oleoyl-sn-glycero-3-phosphate (POPA) (sodium salt), 12:0 PA 1,2-dilauroyl-sn-glycero-3-phosphate (DLPA) (sodium salt), 14:0 PA 1,2-dimyristoyl-sn-glycero-3-phosphate (DMPA) (sodium salt), or a combination thereof.

The at least one neutrally charged phospholipid and the at least one negatively charged phospholipid are combined at any suitable mole ratio to provide the desired fusion rate and/or overall charge of the lipid vesicle. In some embodiments, the mole ratio of neutrally charged phospholipids to negatively charged phospholipids is between about 5:1 and about 1:1. For example, in one preferred embodiment, the lipid composition of an anionic lipid vesicle includes DOPC:POPA at a 3:2 mole ratio. In another preferred embodiment, the lipid composition of an anionic lipid vesicle includes DPPC:POPA at a 3:2 mole ratio. In certain embodiments, the mole ratio of neutrally charged phospholipids to negatively charged phospholipids provides an overall negative charge that prevents the lipid vesicles from fusing together and/or facilitates vesicle-to-cell fusion in the liver. By preventing lipid vesicle fusion, particularly while transiting in circulating blood, the neutral to negatively charged phospholipid mole ratio also reduces or eliminates unwanted systemic drug release.

In some embodiments, the lipid vesicles include a diameter of up to 150 nm, between 50 nm and 150 nm, between 70 nm and 150 nm, or any combination, sub-combination, range, or sub-range thereof. More specifically, in one embodiment, the lipid vesicles include a diameter of between 50 nm and 150 nm. In another embodiment, having a formulation according to one or more of the embodiments disclosed herein and a diameter of between 50 nm and 150 nm prevents or substantially prevents the lipid vesicle from crossing the blood-brain-barrier. In a further embodiment, when administered to a subject, this combination of formulation, charge, and size targets the lipid vesicles to the liver cells while preventing or substantially preventing entrapment of the lipid vesicle in lungs and/or organs other than the liver.

The PDE inhibitor includes any PDE inhibitor suitable for encapsulation by the lipid vesicle and/or targeted PDE inhibition in the liver. In some embodiments, the PDE inhibitors are lipophilic. Additionally or alternatively, in some embodiments, the PDE inhibitors are selective inhibitors. For example, in one embodiment, the PDE inhibitor includes one or more PDE 4 inhibitors, such as, but not limited to, rolipram, apremilast, crisaborole, roflumilast, cilomilast, piclamilast, ibudilast, and/or lirimilast. In another embodiment, the PDE inhibitor includes rolipram, a selective PDE-4 inhibitor also known as 4-(3-Cyclopentyloxy-4-methoxy-phenyl) pyrrolidin-2-one, which has the structure shown below:

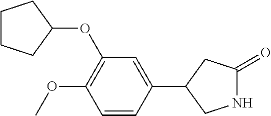 [SMILES: COc1ccc(C2CNC(=O)C2)cc1OC1CCCC1]

In some embodiments, the lipophilic properties of the PDE inhibitor, such as rolipram, which has a log P value of 2.51, permit and/or facilitate incorporation thereof into a membrane-compartment of the lipid vesicle. The incorporation of the rolipram or other lipophilic PDE inhibitor into the membrane-compartment of the lipid vesicle provides increased and/or longer retention as the lipid vesicles circulate in the blood. Increased and/or longer retention of the PDE inhibitors decreases or eliminates free systemic circulation thereof, which decreases or eliminates crossing of the blood-brain-barrier and/or the negative side effects previously associated with PDE inhibitors.

In certain embodiments, a lipid to drug ratio is selected to provide desired, increased, and/or maximal encapsulation efficiency of effective PDE inhibitor levels. For example, in one embodiment, the lipid to rolipram ratio is between about 10:1 and about 30:1 mg of lipid per mL of buffer. In another embodiment, the encapsulation efficiency of the drug in the lipid vesicle is at least 50%, at least 60%, at least 70%, at least 75%, at least 80%, or any combination, sub-combination, range, or sub-range thereof. As will be appreciated by those skilled in the art, the lipid to drug ratio and/or encapsulation efficiency may vary depending upon the specific combination of lipids and PDE inhibitors, and is not limited to the ranges provided above.

The presently-disclosed subject matter also includes a complex for PDE inhibitor delivery. The complex includes one or more of the PDE inhibitors discussed above encapsulated and/or entrapped in one or more of the lipid vesicles discussed above. In some embodiments, the complex also includes one or more excipients. In one embodiment, the one or more excipients include an excipient-emulgent. In another embodiment, the one or more excipients stabilize the PDE inhibitor within the aqueous compartment of the lipid vesicle. In a further embodiment, the one or more excipients extend a shelf-life of the lipid vesicle-PDE inhibitor complex and/or increase water solubility of the PDE inhibitor within the lipid vesicle, which may increase a concentration of PDE inhibitor entrapped in the lipid vesicles and/or the amount of PDE inhibitor delivered to the liver. Suitable excipients include, but are not limited to, sucrose octaacetate (SOA), sucrose acetate isobutyrate (SAIB), polysorbate 80, polyoxyl-15-hydroxystearate, cyclodextrins (HP-CD and SBE-CD), γ-cyclodextrin, hydroxypropyl-γ-cyclodextrin, or a combination thereof. For example, the excipient may include sucrose octaacetate (SOA) added to the buffer in the aqueous compartment to increase rolipram solubility and stability.

The presently-disclosed subject matter further includes methods involving administering one or more phosphodiesterase (PDE) inhibitors to a subject in need thereof. In some embodiments, a method of treating a disease includes administering to a subject in need thereof a therapeutically effective amount of PDE inhibitor encapsulated in a carrier system. In some embodiments, a method of reducing side effects of one or more PDE inhibitors includes administering to a subject in need thereof a therapeutically effective amount of PDE inhibitor encapsulated in a carrier system. The PDE inhibitor includes any suitable PDE inhibitor disclosed herein. For example, in one embodiment, the PDE inhibitor includes a PDE4 inhibitor and/or a biologically active analogue thereof. In another embodiment, the PDE4 inhibitor includes rolipram and/or a biologically active analogue thereof. In a further embodiment, the carrier system includes one or more of the lipid vesicles disclosed herein. The PDE inhibitor encapsulated in the carrier system may be administered to the subject by any suitable method of administration, including, but not limited to, intravenous administration (IV).

In some embodiments, in contrast to existing liposomal drug delivery systems that prolong vesicle circulation time to increase the duration of therapeutic effect, the carrier system disclosed herein is arranged and disposed to deliver and/or target the PDE inhibitor to the subject's liver. Delivering and/or targeting the PDE inhibitor to the subject's liver provides and/or facilitates rapid sequestration of the PDE inhibitor by the liver, which reduces the duration and amount of PDE inhibitor that circulates systemically. In one embodiment, after reaching the liver, the encapsulated PDE inhibitor remains therein where it exerts its therapeutic effect. In another embodiment, encapsulation of the PDE inhibitor in the carrier system and/or targeting of the PDE inhibitor to the subject's liver prevents or substantially prevents the PDE inhibitor from crossing the blood-brain-barrier. In a further embodiment, preventing or substantially preventing the PDE inhibitor from crossing the blood-brain-barrier decreases or eliminates adverse side effects of PDE inhibitors, such as, but not limited to, emesis, nausea, or a combination thereof.

By decreasing or eliminating the adverse side effects of PDE inhibitors, the methods disclosed herein provide and/or facilitate administration of one or more PDE inhibitors for treatment of diseases such as, but not limited to, liver inflammation, alcoholic liver disease, alcoholic hepatitis with or without superimposed cirrhosis, non-alcoholic steatohepatitis (NASH), or a combination thereof. For example, the instant inventors have studied the pathogenic role of PDE4 in regulating hepatic TNF production, hepatic inflammation, and liver injury, and demonstrated a significant up regulation of hepatic PDE4 expression caused by alcohol feeding in mice. The instant inventors have also observed that cyclic AMP (cAMP) decreases when isolated cells are exposed to alcohol, and is associated with an increase in pro-inflammatory cytokine levels. Experiments in which cellular cAMP concentrations were increased attenuated this increase in proinflammatory cytokines. Furthermore, the instant inventors have observed an increase in PDE4B in the decreased cAMP concentrations in alcohol-exposed cells. In view thereof, without wishing to be bound by theory, it is believed that altered PDE4B and cAMP metabolism cause abnormal cytokine (e.g., TNF and IL-10) production/activity, which play a critical role in the development and perpetuation of diseases such as ALD. Accordingly, as the instant inventors have demonstrated that PDE inhibitor-mediated inhibition of PDE4 significantly down-regulates LPS-inducible TNF, targeted delivery of PDE inhibitors (e.g., rolipram a PDE 4B inhibitor) to the liver provides correction of dysregulated cytokine production to treat one or more of the diseases disclosed above without the side effects resulting from PDE inhibitors crossing the blood-brain-barrier and entering the CNS.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

The following examples describe the development of lipid vesicle carriers that target PDE inhibitors to the Kupffer cells in the liver while limiting drug access to the CNS to reduce or eliminate side effects of the drug. More specifically, the instant inventors encapsulated PDE inhibitors in NLs or FLVs that are specifically sized and formulated to target Kupffer cells in the liver. To enhance the PDE inhibitor entrapment efficiency of nanoliposomes (NLs) and targeting of the therapy to the liver, three important factors were considered: 1) specific lipid formulation, vesicle size, and charge (−) were determined to target the NL-PDE inhibitor to liver Kupffer cells; 2) an optimal lipid to drug ratio was determined to achieve maximal encapsulation efficiency of effective PDE inhibitor levels; and 3) an amount of the excipient added to the buffer in the aqueous compartment was determined to increase PDE inhibitor solubility and stability.

### Example 1: Effects of Chronic Alcohol Exposure on cAMP Metabolism and cAMP Effects on Cytokines

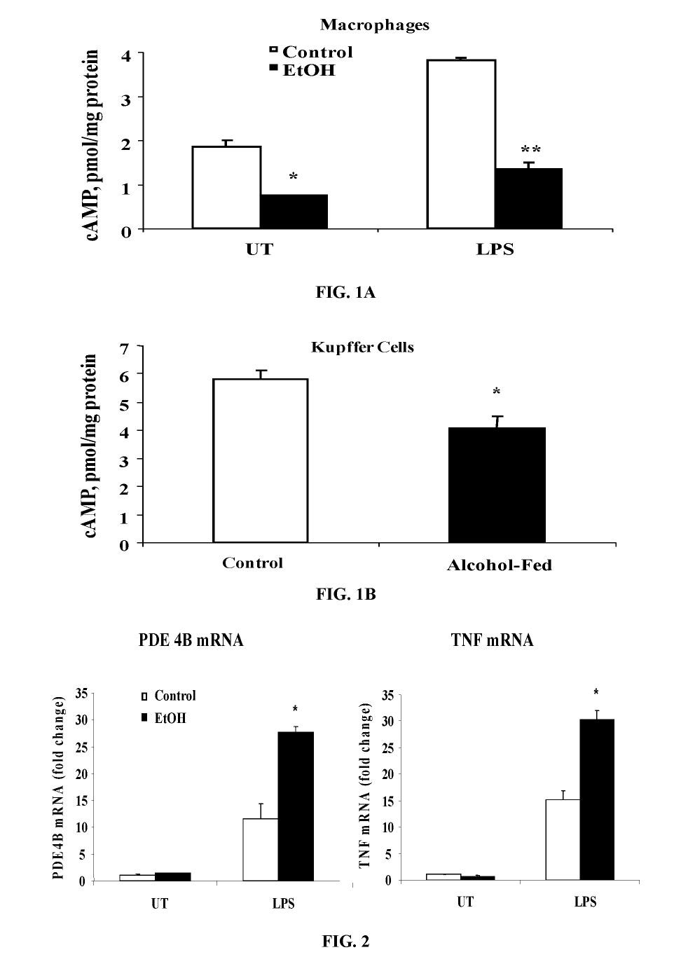

Studies in monocytes have shown that cAMP plays an important role in regulating TNF expression, and that the elevation of cellular cAMP suppresses TNF production. Therefore, the instant inventors evaluated (1) the effects of chronic ethanol exposure on the cellular levels of cAMP, and (2) TNF expression in monocytes in vitro and in rat primary hepatic Kupffer cells obtained from a clinically relevant enteral alcohol feeding model of alcoholic liver disease (ALD). The results indicated that chronic ethanol exposure significantly decreased cellular cAMP levels in both LPS-stimulated and un-stimulated monocytes (both in mouse macrophages—RAW 264.7 and Kupffer cells from rats that were chronically and intragastrically fed alcohol) (FIGS. 1A-B). Consistent with these decreased cAMP levels, ethanol led to an increase in LPS-inducible TNF mRNA expression (FIG. 2) and TNF protein, without any change in the TNF mRNA stability (data not shown). Enhancement of cellular cAMP with dbcAMP abrogated LPS mediated TNF expression in ethanol treated cells (data not shown). Importantly, cAMP did not affect LPS-inducible NFkB activation, but significantly decreased its transcriptional activity (data not shown). Taken together, these data strongly suggest that ethanol can synergize with LPS to up-regulate the induction of TNF gene expression and consequent TNF overproduction by decreasing the cellular cAMP levels in monocytes/macrophages.

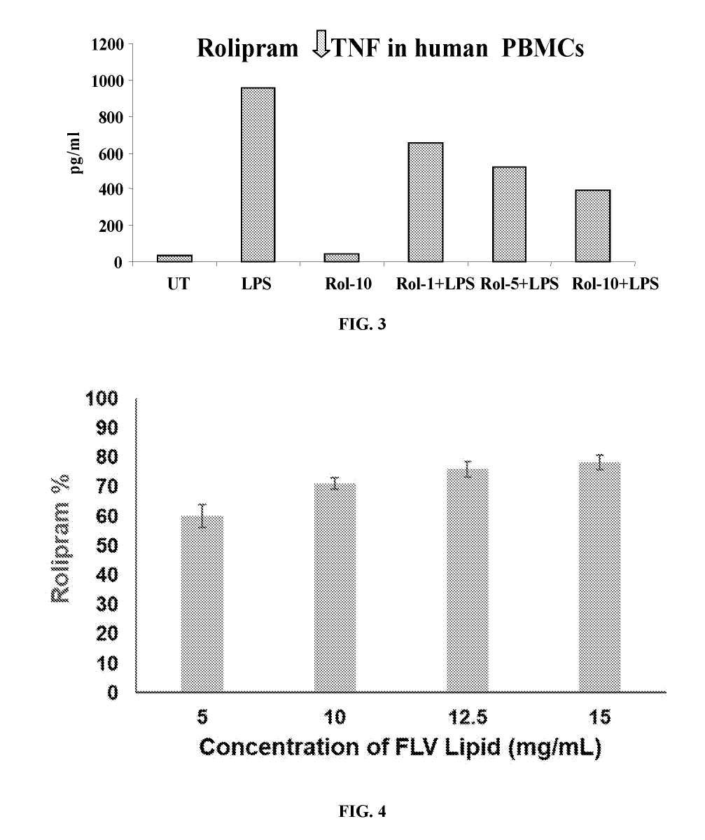

The effect of chronic ethanol exposure on PDE4 gene expression and activity in human THP-1 and mouse RAW macrophages was evaluated next, as a mechanism for the decreased intracellular cAMP levels and the increased LPS-stimulated TNF.⁴ PDE4 (predominantly present in monocytes) degrades and inactivates cAMP, with PDE4B playing a critical role in LPS signaling. It was shown that chronic alcohol increased LPS-inducible PDE4B mRNA expression (FIG. 2). Moreover, the specific PDE4 inhibitor, rolipram, significantly downregulated TNF mRNA (data not shown) and protein in human PBMCs stimulated with LPS in a dose-dependent manner (FIG. 3). These data further support the potential role of a PDE4 inhibitor as a novel anti-inflammatory therapy for ALD.

### Example 2: Development of FLV-Based Carrier to Reduce Rolipram Side Effects

To evaluate the ability of the FLVs to retain rolipram in an aqueous system, 0.75 mg/mL of rolipram was loaded into the lipid bilayer and internal liquid compartment of FLVs using four concentrations of lipid: 5, 10, 12.5, and 15 mg/ml. The rolipram encapsulation efficiency was quantified. Results showed that the initial encapsulation efficiency of rolipram increased as the amount of lipid increased up to about 80% (FIG. 4). These studies demonstrate that the encapsulation efficiency of FLVs-rolipram is high.

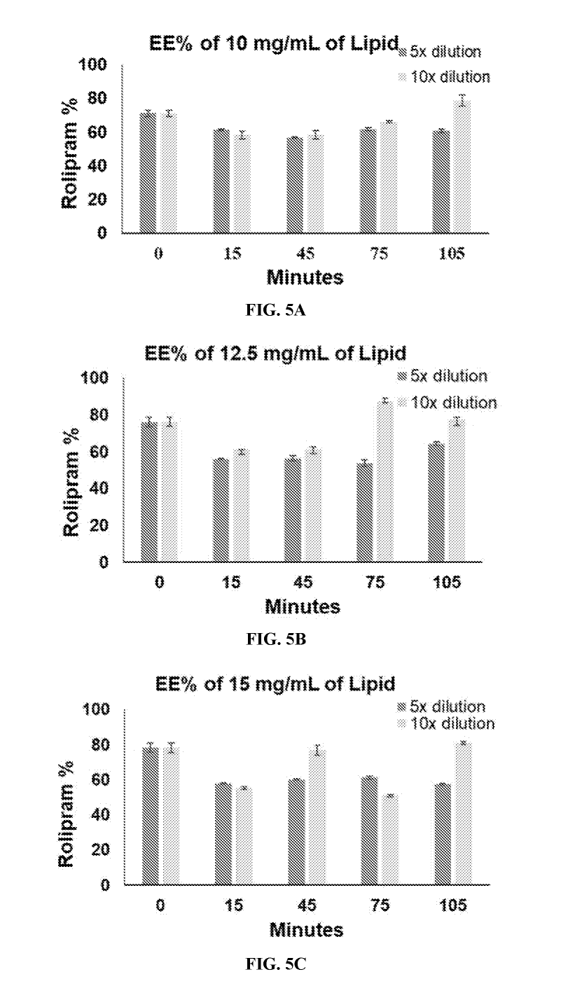

To evaluate the effect of dilution on encapsulation efficiency of FLVs-rolipram, different concentrations of FLV lipid (10, 12.5 and 15 mg/ML) were hydrated with the same amount of rolipram (0.75 mg/mL) and then diluted in water 5 and 10 times. Rolipram levels in the external buffer compartment were measured over a 90 min period. The results showed that after dilution in a closed system FLVs released rolipram over time but levels in buffer never were less than 50% of the total drug used (FIGS. 5A-C). The results also showed that FLVs retain rolipram in a reasonable amount when diluted, a property that favors low circulating levels of free drug in vivo, and extends the time frame of uptake by liver of FLVs-rolipram complexes out of the circulation.

### Example 3: Stability of NLs and Effect of Excipients

Water-soluble non-polar excipients sucrose octaacetate (SOA) and sucrose acetate isobutyrate (SAIB) were added to the aqueous compartment of NLs or FLVs to stabilize rolipram in the vesicle's aqueous compartment. SOA and SAIB are emulgents that decrease the interaction of molecules in solution. SOA and SAIB did not significantly alter the rolipram encapsulation efficiency (FIG. 6), and thereby, would not affect the load of drug delivered to the liver. SOA and SAIB did not interfere with FLV formation. The mean particle size of FLVs with SOA, SAIB or without excipient were 132±51.5 nm, 134.9±57.2, and 123.6±52.9 nm, respectively.

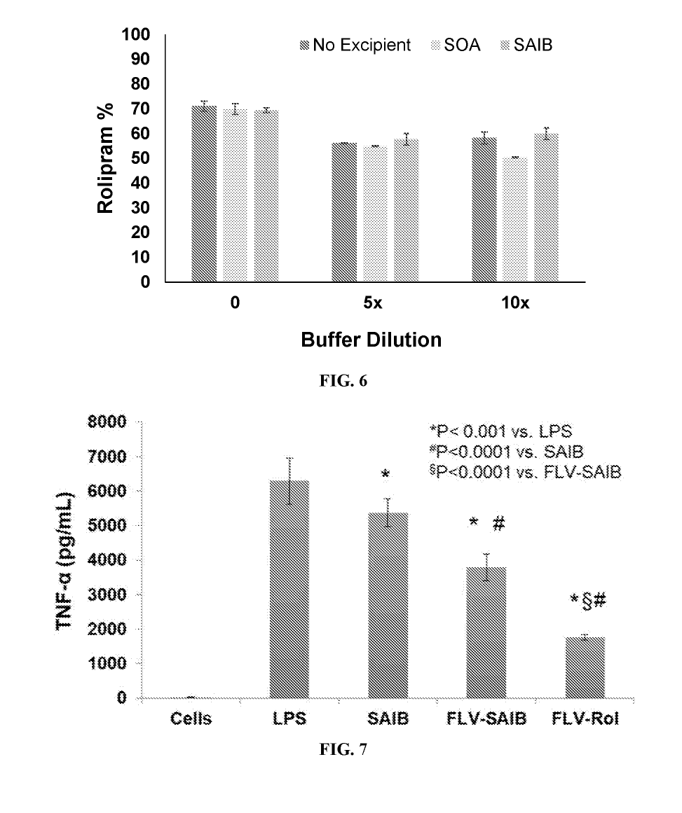

The effect of adding SAIB (0.5 mg/mL) to the vehicle was tested in vitro using RAW cells pulsed with LPS (100 ng/mL) and evaluated for TNF production. Rolipram (0.75 mg/mL) encapsulated in of 10 mg/mL of lipid without SAIB was used as a control. SAIB reduced the effect of LPS, and also, FLVs (10 mg/mL) with SAIB without rolipram further reduced TNF production after LPS exposure (FIG. 7). These results suggest that the excipient interfered with the effect of LPS and that SAIB with FLVs had a significant effect in reducing TNF production.

### Example 4: Fusion Rate of Vesicles Loaded with Rolipram

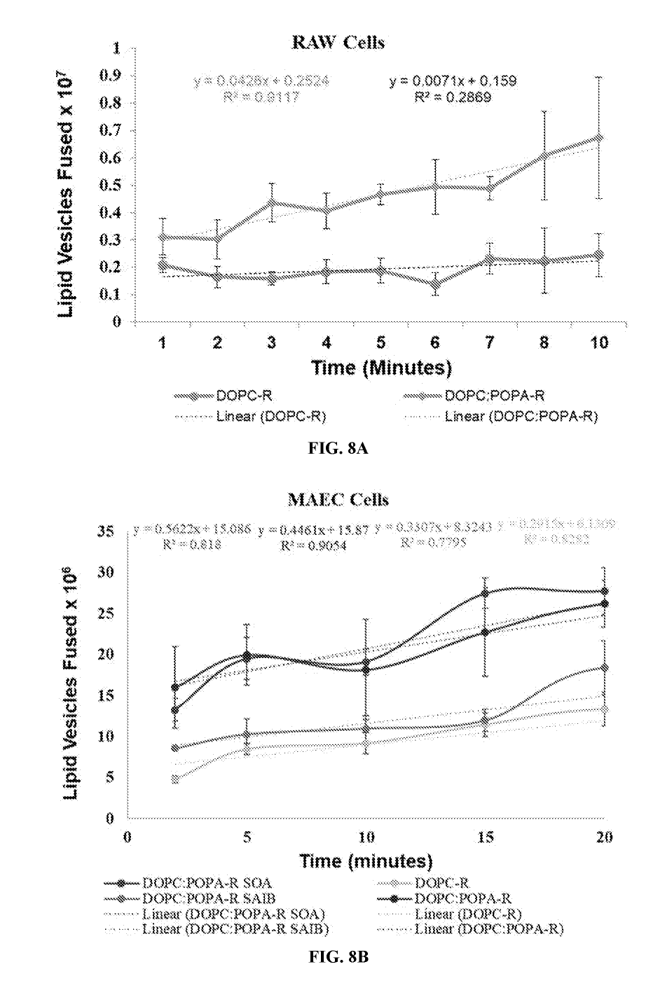

Studies quantifying the fusion rate of vesicles loaded with rolipram to RAW or mouse aortic endothelial cells (MAECs) were performed. In the first experiment, the fusion rate of lipid vesicles to RAW cells using a highly fusogenic vesicle formulated with DOPC/POPA was compared to less fusogenic vesicle formulated with DOPC (FIG. 8A). The results showed that FLVs formulated with DOPC:POPA had a much higher fusion rate than DOPC formulated vesicles. In the second experiment, the fusion rate of FLVs to MAECs using the excipients sucrose octaacetate (SOA) or SAIB and the same vesicle lipid formulations as in the experiment above were performed. The results showed that SOA enhanced the fusion rate of DOPC/POPA FLVs compare to vesicles with SAIB, FLVs with no excipient or DOPC vesicles (FIG. 8B). The above results suggest that the rate of FLV fusion appears to be different between different cell types. Also, SOA an excipient used to enhance the stability of FLVs-rolipram during storage appears to augment the fusion rate of FLVs.

### Example 5: In Vivo Distribution and Localization of FLVs

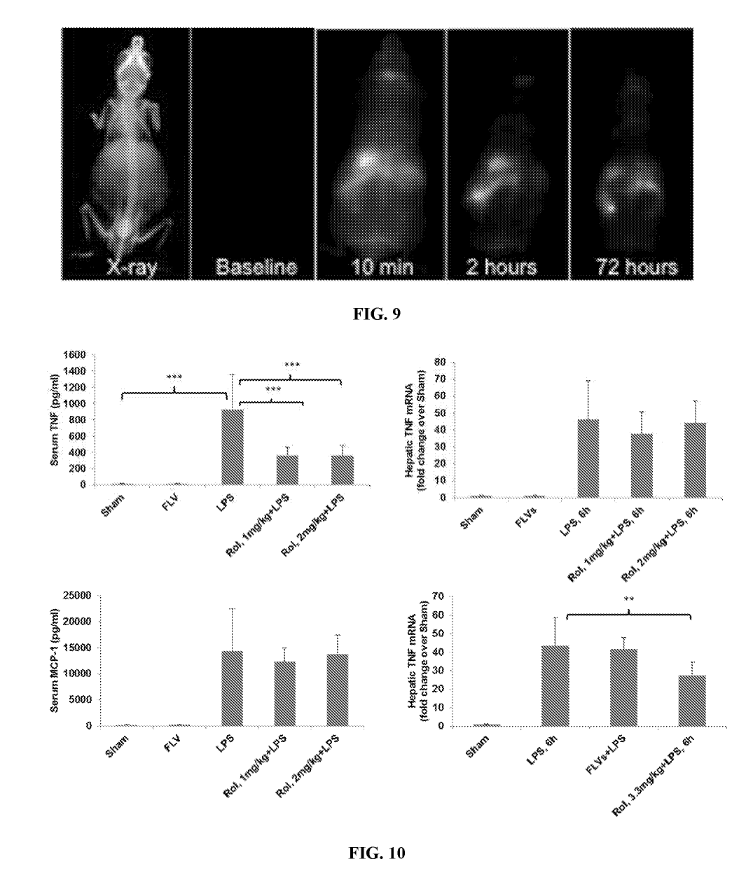

To document the major uptake of FLVs by the liver but not CNS, FLVs were labeled with DiR (a lipophilic near-infrared dye) and infused i.v. into nude mice. The FLVs were tracked in vivo for 96 hours using a Kodak Image Station 4000. FIG. 9 shows the results for the i.v. infused DiR-labeled FLVs. After, 10 min of the initial infusion, the FLVs were mostly localized in liver, (for reference see x-ray image); by 2 h, the majority of FLVs were in the liver, with negligible traces in the brain or other major organs. After 72 h, a significant amount of signal remained in liver suggesting the presence of DiR-labeled FLVs or DiR-labeled FLV-lipid residue. Based on the these experiments it appears that the instant FLV delivery system will prevent side effects by targeting liver, and thus, limiting circulating levels of free rolipram from reaching the CNS to induce emesis.

### Example 6: Structure-Activity Relationship Analysis of FLV Lipid Toxicity

An in silico analysis of the potential toxicity of lipids used in the instant FLV formulation was performed by Dr. A. R. Cunningham, the developer of the software for the cat-structure-activity relationship (cat-SAR). The cat-SAR program estimates the toxicological properties of chemicals, based on information from previously tested compounds. The method has been described in detail in several peer-reviewed publications.³⁰⁻³² The models are built for specific toxicological endpoints (e.g., carcinogenicity or genotoxicity) and describe the chemical substructures that differentiate between active and inactive chemicals for the endpoint of interests (e.g., carcinogens and non-carcinogens).

Table 1 lists the predicted toxicity values for each lipid as a probability of activity of all possible metabolites. The Cut-Off point values correspond to the Validation Results and are used to separate the probability of activity values to “positive” and “negative” calls. The first value is from a model with equal sensitivity and specificity and the second value is from a model with the best overall concordance between experimental and predicted results. In order to assess the toxicological potential of the FLV lipids DOPC and POPA, the cat-SAR models were adjusted for a balance between sensitivity and specificity. The results showed that DOPC and POPA were inactive for Salmonella mutagenicity, carcinogenic potency for rat cancer, human developmental toxicity, MCF-7 Relative Proliferate Effect (ESCREEN), and FDA National Center for Toxicological Research Estrogen Receptor Binding (NCTER ER). However, DOPC and POPA were positive for mouse cancer; however, according to Dr. Cunningham, a positive mouse cancer finding is muted in the setting of negative findings for rat cancer and Salmonella mutagenicity. The rationale is that a negative prediction of mutagenicity in the Salmonella model goes against the notion of a metabolite being a mutagenic carcinogen.

TABLE 1

| | | DOPC | POPA |
|---|---|---|---|
| | CUT-OFF | Pr(activity)/ | Pr(activity)/ |
| Prediction Overview Model | Value | Activity call | Activity call |
| Salmonella, NTP | 0.40 | 0.07/Inactive | 0.08/Inactive |
| Version date: Apr. 17, 2009 | | | |
| Model parameters: (3/0.65/0.9) | | | |
| Rat Cancer, CPDB | 0.73 | 0.65/Inactive | 0.67/Inactive |
| Version date: May 7, 2010 | | | |
| Model parameters: (2/0.70/0.85) | | | |
| Mouse Cancer, CPDB | 0.64 | 0.79/Active | 0.79/Active |
| Version date: Jun. 7, 2010 | | | |
| Model parameters: (4/0.65/0.80) | | | |
| Human Developmental Toxicity | 0.27 | 0.06/Inactive | 0.06/Inactive |
| Version date: Apr. 22, 2009 | | | |
| Model parameters: (3/0.85/0.85) | | | |
| Relative Proliferative | 0.86 | 0.68/Inactive | 0.72/Inactive |
| Effect, ESCREEN | | | |
| Version date: Jun. 5, 2009 | | | |
| Model parameters: (3/0.85/0.65) | | | |
| Estrogen Receptor | 0.83 | 0.37/Inactive | 0.37/Inactive |
| Binding, NCTRER | | | |
| Version date: Nov. 17, 2009 | | | |
| Model parameters: (3/0.80/0.95) | | | |

### Overview of Examples 7-9

Examples 7-9 were aimed at developing a novel fusogenic lipid vesicle (FLV) delivery system that specifically targeted effective levels of rolipram to the liver, while limiting access to the CNS and side effects. The experiments focused on determining an optimal low-dose of 1^{st} generation FLVs-rolipram complexes to inhibit LPS-induced cytokine expression in the liver, without affecting brain PDE4 activity.

The initial experiments were performed in rats, in five distinct groups (n=10 per group): (1) a sham group receiving an injection of PBS; (2) a group receiving an injection of empty FLVs intra-peritoneal (i.p.); (3) a group receiving an injection of LPS (i.p. 1 mg/kg body weight (bw)); and three groups receiving injections of first generation FLVs containing either 1, 2 or 3.3 mg/kg bw of Rolipram. FLV-rolipram was administered 4 h before LPS injection. Six hours after LPS injection, blood samples were collected, the animals were euthanized, and the liver and brain were harvested. Serum cytokine levels were measured by ELISA kits and tissue TNF mRNA levels were assessed by real time PCR.

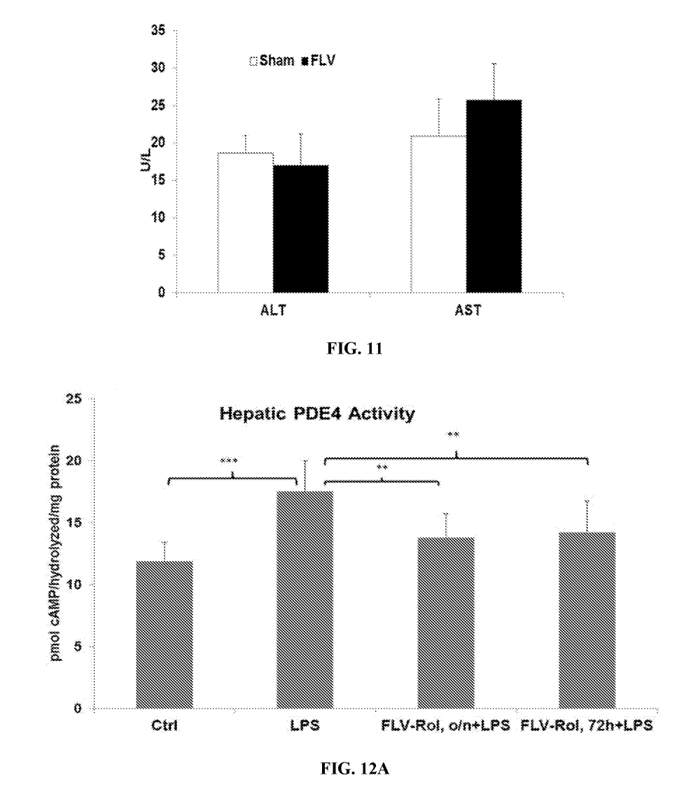

The data showed that serum TNF levels were decreased by all doses of FLVs-rolipram. However, monocyte chemoattractant protein 1 (MCP-1) levels were not affected. Hepatic TNF mRNA levels were not decreased by 1 and 2 mg/kg bw Rolipram dose; however the 3.3 mg/kg bw dose significantly attenuated hepatic TNF mRNA (FIG. 10). Importantly, administration of FLVs alone did not change the serum cytokine levels or serum liver enzyme levels of Alanine Aminotransferase (ALT) and Aspartate Aminotransferase (AST), demonstrating that the FLV-lipid dose used was not hepatotoxic (FIG. 11). Therefore, a dose of FLVs-rolipram (3.3 mg/kg bw) was used in all experiments. Additionally, the rolipram encapsulation efficiency of FLVs was further improved by increasing the lipid content of DOPC/POPA at a mole ratio of 3.2. This formulation retained the fusogenic characteristics of vesicles, and demonstrated enhanced encapsulation efficiency of drug (see Example 2).

Additional single dose studies were conducted in C57Bl/6 mice using an improved FLV formulation and a FLVs-rolipram dose of 3.3 mg/kg bw. The goal of these Examples was to determine the longevity of a single dose of FLVs-rolipram in response to an LPS stimulus administered 12 h or 72 h post-therapy. Blood, brain and liver tissues were collected 3 h after LPS. Assessment of liver PDE4 specific enzymatic activity demonstrated that FLVs-rolipram effectively decreased PDE4 activity compared to the LPS control group; this effect was maintained up to 72 h post FLVs-rolipram administration (FIG. 12A). These data suggest that the FLVs-rolipram in the liver is perhaps entrapped in the FLV-lipid incorporated into cell membranes, yet is still effective in inhibiting PDE4 activity. Notably, an examination of brain PDE4 activity showed that the FLVs-rolipram did not affect the increase in LPS-inducible PDE4 activity in the brain (FIG. 12B). PDE4 activity was measured using a PDE4 activity kit as described previously.⁴

### Example 7: Effects of a Single Dose of FLVs-Rolipram

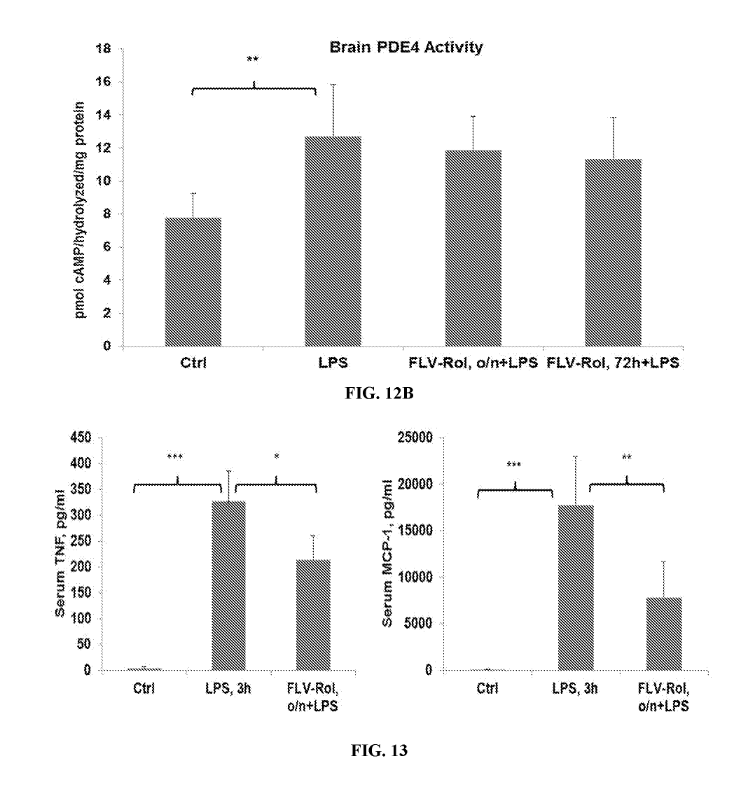

To evaluate the longevity of a single FLVs-rolipram injection on LPS-inducible serum cytokines, the Luminex 100 IS system (Luminex Corp., Austin, Tex.) was used. The advantage of the Luminex technology is that it allows the simultaneous measurement of different analytes in a single sample using as little as 25 μl or less. IFN-γ, IL-1β, L-6, IL-10, IL-12p40, MCP-1 and TNF were quantified, and it was observed that mice treated with FLVs-rolipram, 12 h prior to an LPS injection, experienced a significant decrease in serum TNF and MCP-1 levels compared to LPS controls (FIG. 13).

### Example 8: In Vitro FLV Efficacy Assay

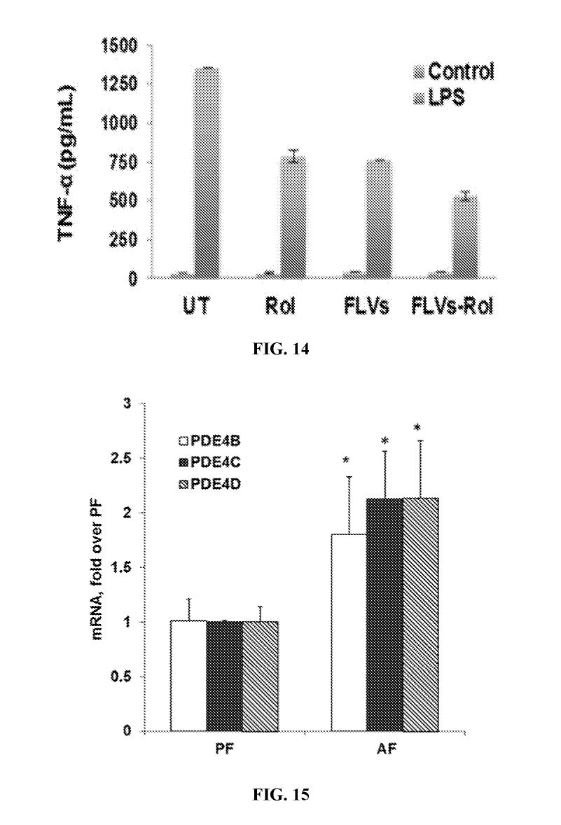

An in vitro screening assay to evaluate the effectiveness of the FLVs-rolipram formulations was developed. The assay uses a murine macrophage cell line (RAW 264.7 cells), which produces high levels of TNF in response to LPS stimulation. The cells were plated in a 24-well plate and pre-treated next day with the improved DOPC/POPA formulated FLVs-rolipram, 4 h before LPS stimulation. After 24 h, TNF protein was measured in cell free supernatant by ELISA kit (FIG. 14). These results indicate that FLVs with entrapped rolipram elicit an effect that reduces TNF production. These findings suggest that this assay would be suitable and functional for both FLV formulation optimization and stability.

### Example 9: Determine Whether PDE4 Inhibition with Low-Dose FLVs-Rolipram Blocks the Development of Alcohol-Induced Liver Injury in a Chronic-Plus-Binge Alcohol-Feeding Model of ALD

A chronic-plus-binge alcohol feeding model of ALD, described by Bin Gao's group,³³ was used in Example 9. In this model—in which the mice develop steatosis, liver injury, and inflammation—the mice are fed the Lieber-DeCarli liquid diet containing 5% ethanol for 10 days; on the 11^{th} day, they are given 31.5% (vol/vol) ethanol gavage, which is similar to the drinking pattern in many alcoholic hepatitis patients that have a background of chronic drinking for many years (chronic) and a history of recent excessive alcohol consumption (binge). This model was used to examine the effect of FLVs-rolipram in attenuating liver injury induced by alcohol. C57Bl/6 mice were pair-fed the Lieber-DeCarli liquid diet (Bio-Serv, Frenchtown, N.J.) as described previously. FLVs-rolipram was administered 12 hours before alcohol gavage. The mice were sacrificed 6 h after alcohol gavage. Control mice, on an isocaloric liquid control diet, were gavaged with maltose dextrin instead of alcohol. The mice were divided into six groups: Group 1 included pair-fed (PF)-mice receiving Lieber-DeCarli liquid control diet (no ethanol); Group 2 mice on the control diet were administered FLVs only (PF+FLVs) 12 h before maltose dextrin gavage; Group 3 mice on the control diet were administered FLVs-rolipram (PF+FLV-Rol) 12 h before maltose dextrin gavage; Group 4 mice on Lieber-DeCarli liquid ethanol diet (5% ethanol) for 10 days followed by alcohol gavage (31.5% vol/vol) (AF); Group 5 mice on Lieber-DeCarli liquid ethanol diet were administered FLVs 12 h (AF+FLV) before ethanol gavage; and Group 6 mice on Lieber-DeCarli liquid ethanol diet were administered FLVs-rolipram (AF+FLV-Rol) 12 h before ethanol gavage.

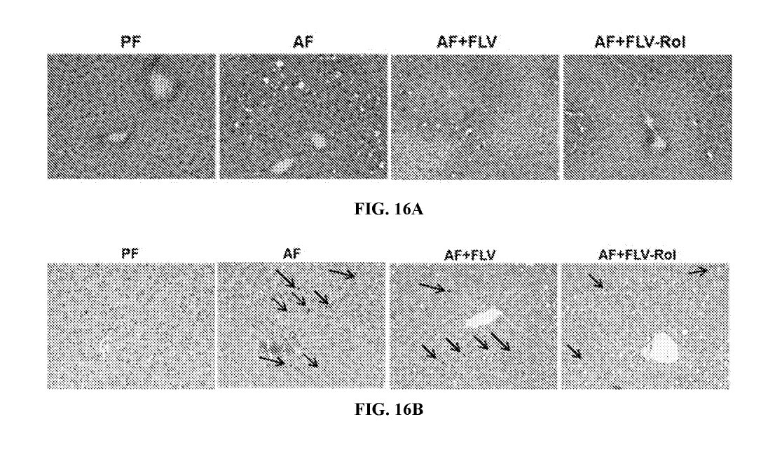

Assays for liver enzymes (ALT, AST), liver caspase 3, TUNEL, CAE, Oil red O staining were performed to document liver injury, steatosis and inflammation. Alcohol induced PDE4 expression changes were assessed by measuring hepatic PDE4 mRNA levels. As expected, ethanol feeding induced a significant upregulation of PDE4B, C and D mRNA levels in the liver (FIG. 15). Importantly, examination of steatosis by Oil Red O staining showed marked attenuation of alcohol induced lipid accumulation in the liver by both FLVs and FLVs-rolipram (FIG. 16A) groups. For the groups given FLVs alone and FLVs-rolipram, lipid accumulation was not affected in PF group (data not shown).

Neutrophil infiltration into the liver was estimated by means of naphthol AS-D chloroacetate esterase (CAE) staining of liver sections. The resulting images demonstrated that FLVs-rolipram reduced neutrophil infiltration in livers induced by alcohol gavage (FIG. 16B).

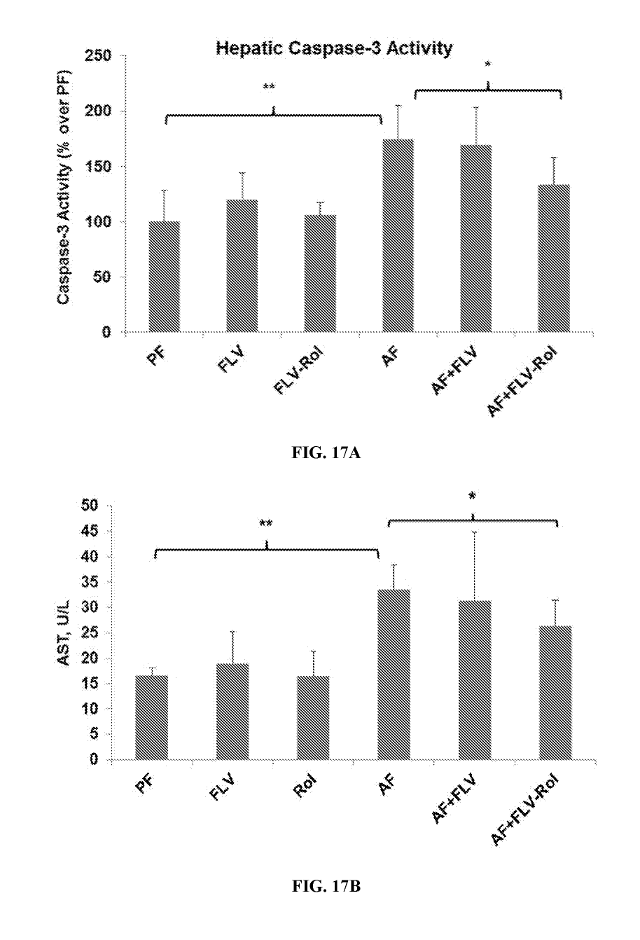

To further evaluate the effect of FLVs-rolipram on alcohol induced liver injury, the Caspase-3 assay was performed on liver lysates using the Caspase-3 kit (Promega Corporation, Madison, Wis.). The data showed that alcohol-gavage resulted in a significant increase in liver caspase-3 activity (FIG. 17A). FLV pretreatment did not affect alcohol induced caspase-3 activity, treatment with FLVs-rolipram significantly decreased it (FIG. 17B). Correlating with the decreased caspase 3 activity, significantly lower levels of serum AST by FLVs-rolipram were observed as compared to alcohol fed (AF) group (FIG. 17B).

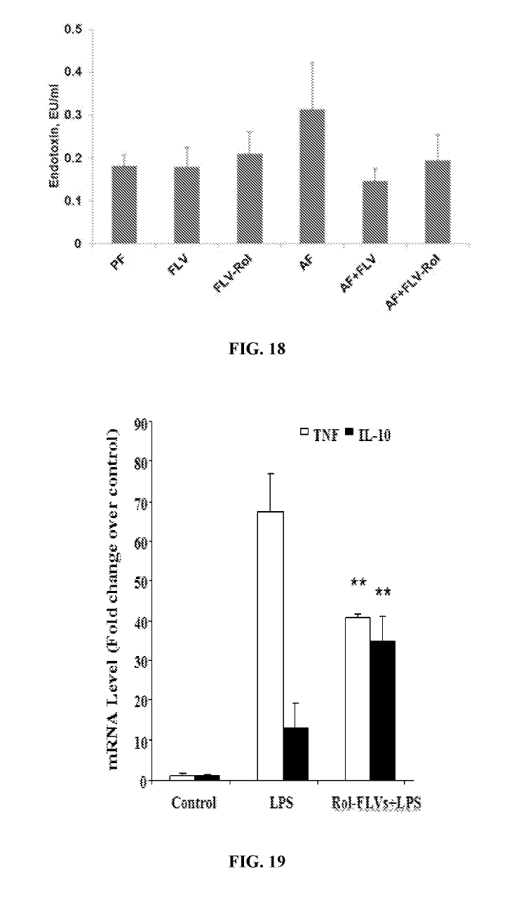

Alcohol consumption causes an increase in gut permeability and endotoxemia which plays a critical role in alcohol mediated liver injury. The effect of FLVs-rolipram on endotoxin levels were evaluated in this model and an increase in endotoxin levels after alcohol-binge was observed (FIG. 18). This effect decreased with both FLVs and FLVs-rolipram, although it did not reach significance. From these studies, it was concluded that the FLV carrier system targeted FLVs-rolipram to the liver and reduced injury in a model of ALD.

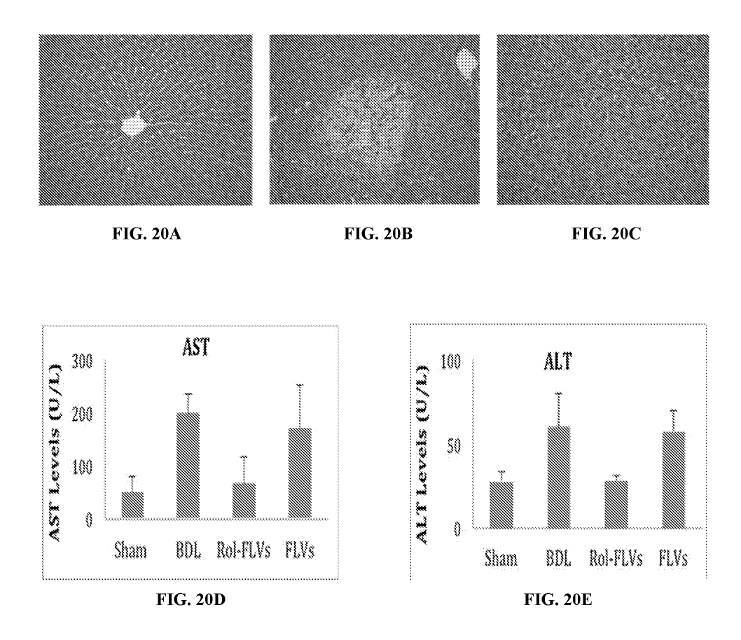

The effect of FLVs-rolipram on LPS-inducible TNF and IL-10 expression in the liver was tested. Wistar rats were injected (i.p.) with FLVs-rolipram (3.3 mg/kg bw) and 4 hours later administered LPS (1 mg/kg bw). FLVs-rolipram significantly inhibited LPS-inducible TNF mRNA and upregulated IL-10 mRNA in the liver (FIG. 19). The same dose of FLVs-rolipram was used in the rat model of cholestatic liver injury (bile duct ligation model). The preliminary data shows that FLVs-rolipram at a lower dose (3.3 mg/kg/body weight) than in previous free rolipram studies (5 mg/kg/body weight) reduced liver injury as demonstrated by H&E staining and the reduced levels of liver enzymes (FIGS. 20A-E). These results demonstrate that the FLVs-rolipram therapy disclosed herein may be clinically effective against liver fibrosis.

### Example 10: Encapsulation of Rolipram in NLs Reduces Side Effects

Safety pharmacology studies were conducted to determine the dose-response relationship of adverse effects observed by rolipram. The primary side effect of rolipram is the inhibition of PDE4 activity in the CNS, which leads to emesis in humans. The mechanism of the emetic response associated with PDE4 inhibitors is thought to produce a pharmacological response analogous to that of presynaptic α2-adrenoceptor inhibition, by elevating intracellular levels of cAMP in noradrenergic neurons. Consequently, PDE4 inhibitors remove an inhibitory mechanism that modulates the release of mediators (5-HT, substance P, noradrenaline) involved in the onset of the emetic reflex. Without wishing to be bound by theory, it is believed that encapsulating rolipram in fusogenic lipid vesicles (FLVs) will limit the drug from reaching the central nervous system (CNS), and thus, attenuate the emetic side effects. Accordingly, the goal of these dose response studies was to examine the effect of rolipram alone or encapsulated in FLVs on the duration of anesthesia.

Since rodents are a non-vomiting species, examining rolipram-induced side effects in rodents is particularly difficult.³⁴⁻³⁶ To circumvent this problem, a behavioral correlate of emesis model in mice was utilized.³⁵ In this model, rolipram is used to reverse the duration of α2-adrenoceptor-mediated xylazine/ketamine-induced anesthesia, which is temporally quantified by the return of the righting reflex. More specifically, using a paired design study, male C57BL/6 mice (25-30 g bw; n=6 per group unless indicated otherwise) were anesthetized with a ketamine (80 mg/kg bw) and xylazine (10 mg/kg bw) mixture administered in a single intraperitoneal (i.p.) injection. The anesthesia mixture was freshly prepared for each set of experiments by mixing 4.8 mL of ketamine, 1.5 mL of xylazine, and 13.7 mL of saline. After 15 min, the mice were placed on a controlled heating pad in the dorsal recumbent position and the duration of anesthesia was determined by timing the return of the righting reflex.

Four days later, the same animals were re-anesthetized with the same dose of ketamine/xylazine, placed in the dorsal recumbent position, injected IV with either free rolipram (1.6, 3.3, and 6.6 mg/kg bw) or FLVs-rolipram (1.6, 3.3, and 6.6 mg/kg bw), and the duration of anesthesia was measured again. A PBS solution containing 0.375 mg/mL of rolipram was used to deliver the 1.6 mg/kg bw of rolipram dose. The concentration of rolipram in PBS solution for the 3.3 and 6.6 mg/kg bw doses was 0.75 mg/mL. The FLVs were made of DOPC and POPA at a 3:2 mole ratio and were prepared using sonication and extrusion. FLV size was quantified by nano-tracking analysis (NTA) using a Particle Metrix system. A PBS solution containing 0.75 mg/mL of rolipram and a dose of 55 mg/kg bw of lipid was used to deliver the 1.6 and 3.3 mg/kg bw rolipram doses. The lipid dose was increased to 110 mg/kg bw for the 6.6 mg/kg rolipram dose.

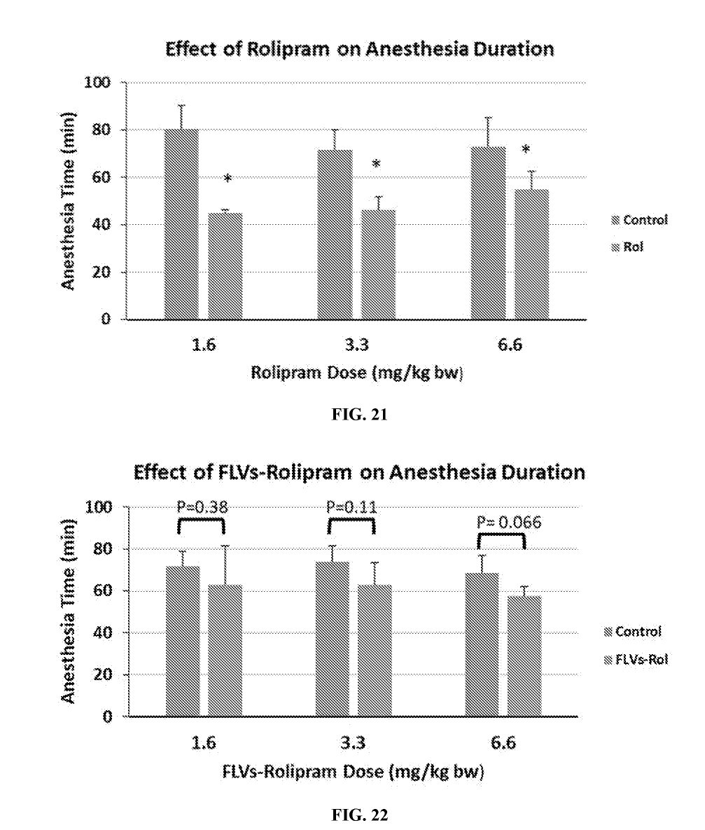

As illustrated in FIG. 21, the mean duration of Xylazine/ketamine anesthesia in the absence and presence of rolipram for the 3 groups was 75.1±4.7 min. Administration of rolipram at all doses (1.6, 3.3 and 6.6 mg/kg bw) significantly reduced the mean anesthesia time for the 3 groups to 48.6±5.5. Turning to FIG. 22, the mean duration of Xylazine/ketamine anesthesia in the absence and presence of FLVs-rolipram for all groups was 71.4±2.7 min. Administration of 1.6 and 3.3 mg/kg bw FLVs-rolipram had no effect on anesthesia time. Although the 6.6 mg/kg bw FLVs-rolipram dose had a modest effect, this effect did not reach significance (P=0.066) as the other doses.

The results of these studies showed that encapsulating rolipram in FLVs prevented a significant shortening of the anesthesia duration for low, target and high doses tested. Accordingly, these results suggest that the side effects caused by free rolipram crossing the blood-brain-barrier and reaching the CNS are ameliorated by FLV encapsulation.

All patents, patent applications, publications, and other published materials mentioned in this specification, unless noted otherwise, are herein incorporated by reference to the same extent as if each individual publication, patent, or patent application was specifically and individually indicated to be incorporated by reference, including the references set forth in the following list:

### REFERENCES

1. Beier J I, Arteel G E, McClain C J. Advances in alcoholic liver disease. Current gastroenterology reports. 2011; 13:56-64.
2. Paula H, Asrani S K, Boetticher N C, Pedersen R, Shah V H, Kim W R. Alcoholic liver disease-related mortality in the united states: 1980-2003. Am. J. Gastroenterol. 2010; 105:1782-1787.
3. Kim W R, Brown R S, Jr., Terrault N A, El-Serag H. Burden of liver disease in the united states: Summary of a workshop. Hepatology. 2002; 36:227-242.
4. Gobejishvili L, Barve S, Joshi-Barve S, McClain C. Enhanced pde4b expression augments lps-inducible tnf expression in ethanol-primed monocytes: Relevance to alcoholic liver disease. Am. J. Physiol. Gastrointest. Liver Physiol. 2008; 295:G718-724.
5. Gobejishvili L, Barve S, Joshi-Barve S, Uriarte S, Song Z, McClain C. Chronic ethanol-mediated decrease in camp primes macrophages to enhanced lps-inducible nf-kappab activity and tnf expression: Relevance to alcoholic liver disease. Am. J. Physiol. Gastrointest. Liver Physiol. 2006; 291:G681-688.
6. Spina D. Pde4 inhibitors: Current status. Br. J. Pharmacol. 2008; 155:308-315.
7. Zidek Z. Adenosine-cyclic amp pathways and cytokine expression. Eur. Cytokine Netw. 1999; 10:319-328.
8. Jin S L, Conti M. Induction of the cyclic nucleotide phosphodiesterase pde4b is essential for lps-activated tnf-alpha responses. Proc. Natl. Acad. Sci. U.S.A. 2002; 99:7628-7633.
9. Jin S L, Lan L, Zoudilova M, Conti M. Specific role of phosphodiesterase 4b in lipopolysaccharide-induced signaling in mouse macrophages. J. Immunol. 2005; 175:1523-1531.
10. Gobejishvili L, Avila D V, Barker D F, Ghare S, Henderson D, Brock G N, Kirpich I A, Joshi-Barve S, Mokshagundam S P, McClain C J, Barve S. S-adenosylmethionine decreases lipopolysaccharide-induced phosphodiesterase 4b2 and attenuates tumor necrosis factor expression via camp/protein kinase a pathway. The Journal of pharmacology and experimental therapeutics. 2011; 337:433-443.
11. Le Moine O, Marchant A, De Groote D, Azar C, Goldman M, Deviere J. Role of defective monocyte interleukin-10 release in tumor necrosis factor-alpha overproduction in alcoholics cirrhosis. Hepatology. 1995; 22:1436-1439.
12. Moore K W, O'Garra A, de Waal Malefyt R, Vieira P, Mosmann T R. Interleukin-10. Annu. Rev. Immunol. 1993; 11:165-190.
13. Hill D B, D'Souza N B, Lee E Y, Burikhanov R, Deaciuc I V, de Villiers W J. A role for interleukin-10 in alcohol-induced liver sensitization to bacterial lipopolysaccharide. Alcohol. Clin. Exp. Res. 2002; 26:74-82.
14. Platzer C, Fritsch E, Elsner T, Lehmann M H, Volk H D, Prosch S. Cyclic adenosine monophosphate-responsive elements are involved in the transcriptional activation of the human il-10 gene in monocytic cells. European journal of immunology. 1999; 29:3098-3104.
15. Endres S, Fulle H J, Sinha B, Stoll D, Dinarello C A, Gerzer R, Weber P C. Cyclic nucleotides differentially regulate the synthesis of tumour necrosis factor-alpha and interleukin-1 beta by human mononuclear cells. Immunology. 1991; 72:56-60.
16. Eigler A, Siegmund B, Emmerich U, Baumann K H, Hartmann G, Endres S. Anti-inflammatory activities of camp-elevating agents: Enhancement of il-10 synthesis and concurrent suppression of tnf production. J. Leukoc. Biol. 1998; 63:101-107.
17. Verghese M W, McConnell R T, Strickland A B, Gooding R C, Stimpson S A, Yarnall D P, Taylor J D, Furdon P J. Differential regulation of human monocyte-derived tnf alpha and il-1 beta by type iv camp-phosphodiesterase (camp-pde) inhibitors. The Journal of pharmacology and experimental therapeutics. 1995; 272:1313-1320.
18. Kambayashi T, Jacob C O, Zhou D, Mazurek N, Fong M, Strassmann G. Cyclic nucleotide phosphodiesterase type iv participates in the regulation of il-10 and in the subsequent inhibition of tnf-alpha and il-6 release by endotoxin-stimulated macrophages. J. Immunol. 1995; 155:4909-4916.
19. De B K, Gangopadhyay S, Dutta D, Baksi S D, Pani A, Ghosh P. Pentoxifylline versus prednisolone for severe alcoholic hepatitis: A randomized controlled trial. World J. Gastroenterol. 2009; 15:1613-1619.
20. Akriviadis E, Botla R, Briggs W, Han S, Reynolds T, Shakil O. Pentoxifylline improves short-term survival in severe acute alcoholic hepatitis: A double-blind, placebo-controlled trial. Gastroenterology. 2000; 119:1637-1648.
21. Kucuktulu U, Alhan E, Tekelioglu Y, Ozekin A. The effects of pentoxifylline on liver regeneration after portal vein ligation in rats. Liver international: official journal of the International Association for the Study of the Liver. 2007; 27:274-279.
22. Peterson T C. Pentoxifylline prevents fibrosis in an animal model and inhibits platelet-derived growth factor-driven proliferation of fibroblasts. Hepatology. 1993; 17:486-493.
23. Desmouliere A, Xu G, Costa A M, Yousef I M, Gabbiani G, Tuchweber B. Effect of pentoxifylline on early proliferation and phenotypic modulation of fibrogenic cells in two rat models of liver fibrosis and on cultured hepatic stellate cells. J. Hepatol. 1999; 30:621-631.
24. Raetsch C, Jia J D, Boigk G, Bauer M, Hahn E G, Riecken E O, Schuppan D. Pentoxifylline downregulates profibrogenic cytokines and procollagen i expression in rat secondary biliary fibrosis. Gut. 2002; 50:241-247.
25. Gobejishvili L, Barve S, Breitkopf-Heinlein K, Li Y, Zhang J, Avila D V, Dooley S, McClain C J. Rolipram attenuates bile duct ligation-induced liver injury in rats: A potential pathogenic role of pde4. The Journal of pharmacology and experimental therapeutics. 2013; 347:80-90.
26. Zhu J, Mix E, Winblad B. The antidepressant and antiinflammatory effects of rolipram in the central nervous system. CNS drug reviews. 2001; 7:387-398.
27. Manji H K, Quiroz J A, Sporn J, Payne J L, Denicoff K, N A G, Zarate C A, Jr., Charney D S. Enhancing neuronal plasticity and cellular resilience to develop novel, improved therapeutics for difficult-to-treat depression. Biol. Psychiatry. 2003; 53:707-742.
28. Fleischhacker W W, Hinterhuber H, Bauer H, Pflug B, Berner P, Simhandl C, Wolf R, Gerlach W, Jaklitsch H, Sastre-y-Hernandez M, et al. A multicenter double-blind study of three different doses of the new camp-phosphodiesterase inhibitor rolipram in patients with major depressive disorder. Neuropsychobiology. 1992; 26:59-64.
29. Ehringer W, Belcher D, Wassall S R, Stillwell W. A comparison of the effects of linolenic (18:3 omega 3) and docosahexaenoic (22:6 omega 3) acids on phospholipid bilayers. Chem. Phys. Lipids. 1990; 54:79-88.
30. Cunningham A R, Cunningham S L, Consoer D M, Moss S T, Karol M H. Development of an information-intensive structure-activity relationship model and its application to human respiratory chemical sensitizers. SAR QSAR Environ. Res. 2005; 16:273-285.
31. Cunningham A R, Consoer D M, Iype S A, Cunningham S L. A structure-activity relationship analysis for the identification of environmental estrogens: The categorical-sar (cat-sar) approach. In: Devillers J, ed. Endocrine disruption modeling. CRC Press; 2009:173-198.
32. Cunningham A R, Moss S T, Iype S A, Qian G, Qamar S, Cunningham S L. Structure-activity relationship analysis of rat mammary carcinogens. Chem. Res. Toxicol. 2008; 21:1970-1982.
33. Bertola A, Mathews S, Ki S H, Wang H, Gao B. Mouse model of chronic and binge ethanol feeding (the niaaa model). Nature protocols. 2013; 8:627-637.
34. Robichaud A, Savoie C, Stamatiou P B, Tattersall F D, Chan C C. Pde4 inhibitors induce emesis in ferrets via a noradrenergic pathway. Neuropharmacology. 2001; 40:262-269.
35. Robichaud A, Stamatiou P B, Jin S L, Lachance N, MacDonald D, Laliberte F, Liu S, Huang Z, Conti M, Chan C C. Deletion of phosphodiesterase 4d in mice shortens alpha(2)-adrenoceptor-mediated anesthesia, a behavioral correlate of emesis. The Journal of clinical investigation. 2002; 110:1045-1052.
36. Robichaud A, Savoie C, Stamatiou P B, Lachance N, Jolicoeur P, Rasori R, Chan C C. Assessing the emetic potential of pde4 inhibitors in rats. Br. J. Pharmacol. 2002; 135:113-118.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A composition for treating liver inflammation, the composition comprising a biologically active phosphodiesterase 4 (PDE4) inhibitor.

2. The composition of claim 1, further comprising a liposome encapsulating the inhibitor rolipram.

3. The composition of claim 2, wherein the liposome is an anionic liposome.

4. The composition of claim 2, wherein the liposome comprises negatively charged phospholipids and neutrally charged phospholipids.

5. The composition of claim 4, wherein a molar ratio of the neutrally charged phospholipids to the negatively charged phospholipids is between 5:1 and 1:1.

6. The composition of claim 4, wherein the negatively charged phospholipids are phosphatidic acid (PA).

7. The composition of claim 4, wherein the neutrally charged phospholipids are phosphatidylcholine (PC).

8. The composition of claim 2, wherein the liposome comprises 1,2-Dioleoyl-sn-glycerol-3-phosphocholine (DOPC) and 1-palmitoyl-2-oleol-sn-glycerol-3-phosphate (POPA).

9. The composition of claim 2, further comprising an excipient.

10. The composition of claim 9, wherein the excipient is sucrose octaacetate.

11. A composition for treating liver inflammation, the composition comprising a phosphodiesterase 4 (PDE4) inhibitor and a liposome, the liposome encapsulating the PDE4 inhibitor.

12. The composition of claim 11, wherein the liposome comprises negatively charged phospholipids and neutrally charged phospholipids.

13. The composition of claim 12, wherein a molar ratio of the neutrally charged phospholipids to the negatively charged phospholipids is between 5:1 and 1:1.

14. The composition of claim 12, wherein the negatively charged phospholipids are phosphatidic acid (PA).

15. The composition of claim 12, wherein the neutrally charged phospholipids are phosphatidylcholine (PC).

16. The composition of claim 11, wherein the liposome comprises 1,2-Dioleoyl-sn-glycerol-3-phosphocholine (DOPC) and 1-palmitoyl-2-oleol-sn-glycerol-3-phosphate (POPA).

17. The composition of claim 11, wherein the liposome is an anionic liposome.

18. The composition of claim 11, wherein the PDE4 inhibitor is a biologically active analogue of rolipram.

19. The composition of claim 18, wherein the PDE4 inhibitor is selected from the group consisting of rolipram, apremilast, crisaborole, roflumilast, cilomilast, piclamilast, ibudilast, and lirimilast.

20. The composition of claim 11, further comprising an excipient.

21. The composition of claim 20, wherein the excipient is sucrose octaacetate.
